(19) United States
(12) Patent Application Publication
(10) Pub. No.: US 2007/0225594 A1
(43) Pub. Date: Sep. 27, 2007

(54) Facilitation of In-Boundary Distortion Compensation

(75) Inventor: Peter Traneus Anderson, Andover, MA (US)

Correspondence Address: ROBERT ALAN EARL, 1816 TENNYSON ROW LANE, MOUNT PLEASANT, SC, 29466, US

(73) Assignee: General Electric Company, Schenectady, NY (US)

(21) Appl. No.: 11/276,228

(22) Filed: Feb. 17, 2006

(51) Int. Cl. A61B 5/05

(52) U.S. Cl. 600/424

(57) ABSTRACT

Described herein are one or more implementations for facilitating the compensation of the effects of distortion caused by “in-boundary distorters.” A “distorter” may be a medical instrument or other element that may distorts a magnetic field. An “in-boundary” distorter is a distorter located inside a boundary of a magnetic field, such as that employed by within an electromagnetic tracking environment.

### BACKGROUND

Medical practitioners, such as doctors, surgeons, and other medical professionals, often rely upon technology when performing a medical procedure, such as image-guided surgery or examination. A tracking system may provide positioning information for the medical instrument with respect to the patient or a reference coordinate system, for example. A medical practitioner may refer to the tracking system to ascertain the position of the medical instrument when the instrument is not within the practitioner's line of sight. A tracking system may also aid in pre-surgical planning.

The tracking or navigation system allows the medical practitioner to visualize the patient's anatomy and track the position and orientation of the instrument. The medical practitioner may use the tracking system to determine when the instrument is positioned in a desired location. The medical practitioner may locate and operate on a desired or injured area while avoiding other structures. Increased precision in locating medical instruments within a patient may provide for a less invasive medical procedure by facilitating improved control over smaller instruments having less impact on the patient. Improved control and precision with smaller, more refined instruments may also reduce risks associated with more invasive procedures such as open surgery.

Tracking systems may be ultrasound, inertial position, or electromagnetic tracking systems, for example. Electromagnetic tracking systems may employ coils as receivers and transmitters. Typically, an electromagnetic tracking system is configured in a so-called “industry-standard coil architecture” (ISCA). ISCA uses three co-located orthogonal quasi-dipole transmitter coils and three co-located quasi-dipole receiver coils. Other systems may use three large, non-dipole, non-co-located transmitter coils with three co-located quasi-dipole receiver coils. Another tracking system architecture uses an array of six or more transmitter coils spread out in space and one or more quasi-dipole receiver coils. Alternatively, a single quasi-dipole transmitter coil may be used with an array of six or more receivers spread out in space.

The ISCA tracker architecture uses a three-axis dipole coil transmitter and a three-axis dipole coil receiver. Each three-axis transmitter or receiver is built so that the three coils exhibit the same effective area, are oriented orthogonally to one another, and are centered at the same point. If the coils are small enough compared to a distance between the transmitter and receiver, then the coil may exhibit dipole behavior. Magnetic fields generated by the trio of transmitter coils may be detected by the trio of receiver coils. Using three approximately concentrically positioned transmitter coils and three approximately concentrically positioned receiver coils, for example, nine mutual-inductance measurements may be obtained. From the nine parameter measurements, a position and orientation calculation may determine position and orientation of the receiver coil trio with respect to the transmitter coil trio with six degrees of freedom.

Many medical procedures involve a medical instrument, such as a drill, a catheter, scalpel, scope, shunt or other tool. Often electromagnetic tracker/receiver assemblies are mounted on surgical instruments to track the position and orientation of the instrument tip in real time during surgery. Medical practitioners, for example, rely on electromagnetic trackers to perform sensitive image-guided surgery. Accuracy of position measurement is important when guiding a precision instrument in a patient without a direct line of sight. Distortion may produce inaccurate position measurements and potential danger to a patient. Thus, a system that reduces inaccurate tracking measurements would be highly desirable. A system that minimizes the effect of distortion on position measurement would be highly desirable.

In an electromagnetic tracking environment, distortion may arise from eddy currents induced in nearby metal objects or from ferromagnetic materials (called simply “distorters” herein). Eddy currents, in turn, generate fields that interfere with the field from the source(s) used for tracking purposes. Examples of such sources include medical instruments with mounted electromagnetic tracker/receiver assembly thereon. Many instruments used in medical activities include distorters (e.g., metal components). Additionally, an environment surrounding a medical instrument or a tracking system may include distorters.

Distortions in the electromagnetic tracking environment may cause the inaccurate tracking in the electromagnetic tracking environment. Conventionally, distortion is minimized by mounting the receiver electromagnetic tracker/receiver assembly on a rigid outrigger arm from the medical instrument. Often, this keeps the receiver assembly away from the most-distorted field region of the medical instrument. However, outrigger arms can make the instrument more difficult to use effectively. Furthermore, some instruments distort too much to use, even with an outrigger arm.

Another approach for compensating for distortion is “field-distortion compensation mapping” techniques. With such mapping techniques, the electromagnetic field in a volume of interest, as distorted by distorters, is defined in advance and used to solve for position and orientation of the item being tracked. U.S. Pat. Nos. 6,400,139 (Khalfin '139) and 6,377,041 (Khalfin '041) are examples of the conventional field-distortion compensation approach.

FIG. 1 of Khalfin '139 shows the conventional approach to field-distortion compensation. As shown, the conventional system compensates for distortion inside a defined volume (e.g., “volume of interest 104” in FIG. 1 of Khalfin '139) when that distortion is caused by distorters (e.g., “source 110” in FIG. 1 of Khalfin '139) located outside of the defined volume. However, these conventional approaches do not compensate for distortion outside a defined volume when that distortion is caused by distorters located inside of the defined volume.

### SUMMARY

Described herein are one or more implementations for facilitating the compensation of the effects of distortion caused by “in-boundary distorters.” A “distorter” may be a medical instrument or other element that may distorts a magnetic field. An “in-boundary” distorter is a distorter located inside a boundary of a magnetic field, such as that employed by within an electromagnetic tracking environment.

This summary itself is not intended to limit the scope of this patent and the appending claims of this patent. Moreover, the title of this patent is not intended to limit the scope of this patent. For a better understanding of the present invention, please see the following detailed description and appending claims, taken in conjunction with the accompanying drawings. The scope of the present invention is pointed out in the appending claims.

### BRIEF DESCRIPTION OF THE DRAWINGS

The same numbers are used throughout the drawings to reference like elements and features.

FIG. 1 illustrates a block diagram representation of an exemplary distortion-compensation system in accordance with one or more implementations described herein.

FIGS. 2A and 2B illustrates use of a distortion-mapping chamber in accordance with one or more implementations described herein.

FIG. 3 illustrates a flow diagram showing a methodological implementation of a distortion-compensation method described herein.

FIG. 4 illustrates a block diagram representation of an exemplary electromagnetic tracking system in accordance with one or more implementations described herein.

FIG. 5 illustrates a flow diagram showing a methodological implementation of a distortion-handling method described herein.

### DETAILED DESCRIPTION

One or more implementations, described herein, facilitates the reduction of and/or actually reduces the effects of distortion caused by a medical instrument or other field-distorting element (i.e., a “distorter”) located inside a boundary of a magnetic field, such as that employed by within an electromagnetic tracking environment. A distorter located inside a boundary of a magnetic field is called an “in-boundary distorter” herein.

One or more of such described implementations takes measurements over a closed surface (i.e., a “boundary”) of a defined volume (e.g., “tracking magnetic field”) and calculates the effects outside the defined volume due to the distortion caused by one or more in-boundary distorters located inside the defined volume.

One or more described implementations may be particular useful, for example, in the realm of surgical navigation using electromagnetic-tracking. In such a realm, the medical practitioner is presented with a three-dimensional visualization of patient's anatomy and has the ability to track the position and orientation of surgical instrumentation during surgery.

Many medical procedures involve a medical instrument, such as a drill, a catheter, scalpel, scope, debrider motor, shunt or other tool. Often electromagnetic-tracker receiver-assemblies are mounted to such medical instruments to track the position and orientation of the instrument tip in real time during a medical procedure, such as surgery. Many instruments used in medical activities include metal components or other such substances that may distort magnetic fields in the electromagnetic tracking system. Distortions in the electromagnetic tracking system may cause the tracking system to be inaccurate.

To avoid the distortion caused by distorters inside the boundary surface of the tracking magnetic field (i.e., “in-boundary distorters”), some approaches have moved the electromagnetic tracker receiver assemblies (and thus the tracking magnetic field) away from the distorters of the medical instrument itself. This is often accomplished by mounting the electromagnetic tracker receiver assembly on an outrigger arm.

However, rather than directly addressing the issue of in-boundary distorters, the outrigger approach avoids the issue by moving the tracking magnetic field away from the distorters. The outrigger approach effectively moves in-boundary distorters outside the tracking magnetic field; thereby converting them into “out-boundary distorters,” which are distorters located outside inside the boundary surface of the tracking magnetic field. However, even with an outrigger, some instruments distort too much to be used effectively. So, the existing outrigger approach does not accommodate for these highly distorting instruments. The outrigger and other existing approaches have not addressed the issue of in-boundary distorters head-on.

One or more described implementations directly addresses the issue of in-boundary distorters head-on. Because of this, electromagnetic tracker receiver assembly may be mounted closer to the distorters of the medical instrument. Consequently, the outrigger may be shortened or eliminated. Doing so results in an improved ergonomic medical instrument.

Using one or more implementations described herein the distortion due to in-boundary distorters (e.g., surgical instrumentation itself) is measured. This measured distortion is accounted for in a field model or mutual-inductance model in the tracking logic of an electromagnetic surgical navigation system. This enables accurate tracking in the presence of in-boundary distorters.

In conventional “field-distortion compensation mapping” techniques—such as those described in U.S. Pat. Nos. 6,400,139 (Khalfin '139) and 6,377,041 (Khalfin '041—the electromagnetic field in a volume of interest, as distorted by out-boundary distorters, is defined in advance and used to solve for position and orientation of the item being tracked. In this conventional approach, all the field distorters are located outside the boundary of a volume of space and field measurements on the boundary are used to calculate the field inside the boundary.

In the one or more implementations described herein, the distorters are located inside the boundary of a volume of space and field measurements on the boundary are used to calculate the field outside the boundary. To be clear, the difference between the conventional “out-boundary distortion” approach and this new “in-boundary distortion” approach of at least one implementation described herein includes at least the following:
a Conventional: out-boundary distorters and calculated field is inside the boundary
New: in-boundary distorters and calculated field is outside the boundary

### Exemplary In-Boundary Distortion-Compensation System Using a Distortion-Mapping Chamber

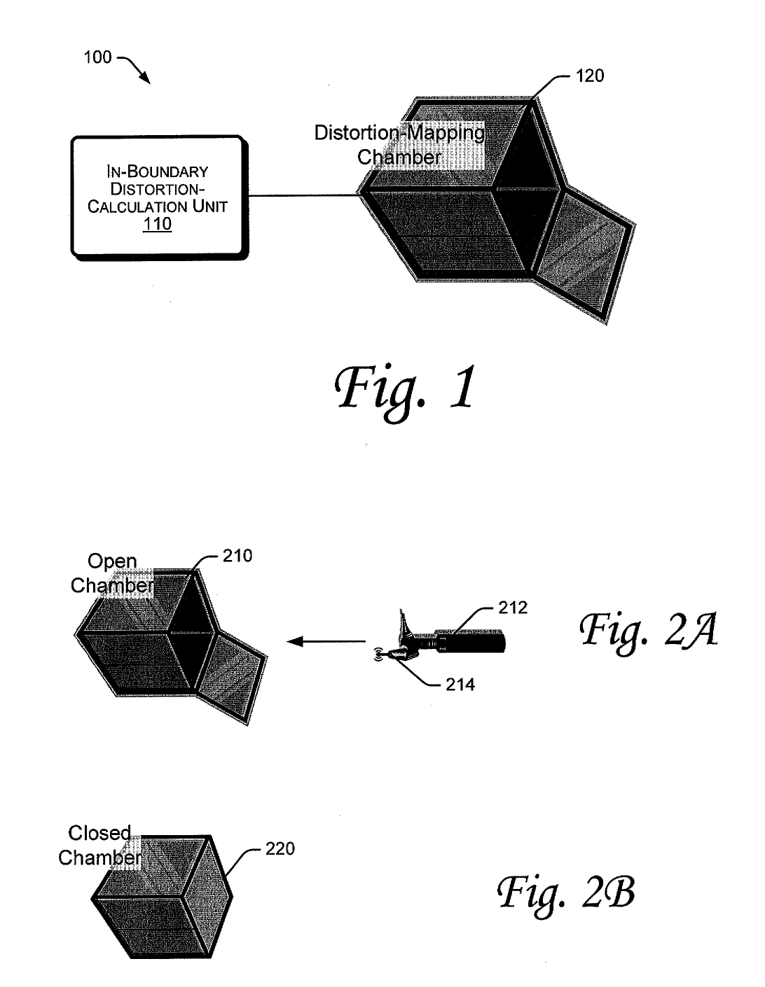

FIG. 1 shows, for the purpose of illustration, an exemplary in-boundary distortion-compensation system 100 for use in measuring and calculating distortion caused by in-boundary distorters. With these measurements and calculations, the accuracy of position and orientation (P&O) measurement of an electromagnetic (EM) tracker may be improved by compensating for the in-boundary distorters used with the EM tracker. The exemplary distortion-compensation system 100 may be part of or separate from an electromagnetic tracking system.

Using the in-boundary distortion-compensation system 100, the distortion caused by one or more in-boundary distorters is measured and mapped. In addition, the distortion-corrected mutual-inductance model may be rapidly calculated in, for example, the operating room just before surgery. Thus, distorters such as medical instruments may be distortion-corrected just before use.

As depicted, the exemplary distortion-compensation system 100 includes an in-boundary distortion-calculation unit 110 connected to a distortion-mapping chamber 120.

The in-boundary distortion-calculation unit 110 may include one or more general-purpose or special-purpose computing system environments or configurations. Examples of well known computing systems, environments, and/or configurations that may be suitable for use include, but are not limited to, personal computers (PCs), server computers, hand-held or laptop devices, thin clients, thick clients, multiprocessor systems, microprocessor-based systems, programmable consumer electronics, wireless phones and equipment, general and special-purpose appliances, application-specific integrated circuits (ASICs), set top boxes, personal digital assistants (PDA), appliances, programmable consumer electronics, network PCs, minicomputers, mainframe computers, distributed computing environments that include any of the above systems or devices, and the like. The distortion-calculation unit 110 may one or more memories, which may be any available processor-readable media that is accessible by distortion-calculation unit 110. The memory may be either volatile or non-volatile media. In addition, it may be either removable or non-removable media.

As shown in FIG. 2A, a distorter 212 (e.g., a medical instrument) is placed the distortion-mapping chamber 210. The distorter 212 has a device-under-test (DUT) 214 attached thereto. An example of a DUT is a transmitter (or receiver) mounted on to the distorter 212.

In at least one implementation, when placed inside the distortion-mapping chamber 210, the distorter 212 is attached to a static arm inside the chamber 210 or alternatively the distorter may be attached to a movable robot arm. FIG. 2B, the distorter 212 is inside the closed distortion-mapping chamber 220. The distorter 212 typically has a tracking-receiver assembly (or some functionally equivalent element) attached thereto. The receiver assembly may be a coil, but the assembly could be any type of magnetic field sensor (such as flux gate, magnetoresistance, Hall Effect, etc.).

The distortion-calculation unit 110 shown in FIG. 1 has one or more components for measuring the magnetic field of the boundary surface of a volume defined by the distortion-mapping chamber 120, to which the unit is connected. The actual field measurements on the boundary surface can be performed with a moving robot arm or with a static array of coils.

The walls of this chamber include receiver coils (alternatively, transmitter coils). Here, the walls of the distortion-mapping chamber 120 are form a “closed and finite” boundary surface. This is the “closed and finite” boundary surface discussed in “Classical Electrodynamics” by John David Jackson, first edition, copyright 1962 by John Wiley and Sons, Inc. On page 19, Jackson states: “The customary Neumann problem is the so-called “exterior problem” in which the volume V is bounded by two surfaces, one closed and finite, the other at infinity.” While Jackson discusses solving the problem using Green's functions, many other solution methods can be used.

For the sake of simplicity, the distortion-mapping chamber is shown in FIGS. 1, 2A, and 2B (which are chambers 120, 210 and 220) as a rectangular box with six flat sides. However, those of skill in the art understand that the other implementations of the chamber may take other shapes. As shown in FIGS. 1, 2A, one side of the chamber (120 and 210) is removeable to permit insertion of the DUT 214. In the chambers shown in FIGS. 1, 2A, and 2B, each side of the chamber (120, 210 and 220) is a printed-circuit board (PCB) covered with approximately 100 to 1000 non-overlapping coils. In other implementations, overlapping coils can also be used. The coils approximate the data needed for the Neumann boundary conditions.

The coil leads may be brought out individually to receiving electronics, such as the distortion-calculation unit 110 shown in FIG. 1. Furthermore, there may be a switch employed next to each coil on the PCB, so that the distortion-calculation unit 110 one or a few coils can be selected at a time. Example switches are: complementary metal-oxide semiconductor (CMOS) switches, junction field-effect transistors (JFETs), bipolar junction transistors (BJTs), JFET-output optoisolators such as the Fairchild Semiconductor type H11F1, diodes, and electromechanical relays (both magnetically-actuated and thermally actuated).

Further still, an integrated circuit may be employed at each coil. One type of suitable integrated circuit may employ a preamp, frequency-shifting analog circuitry, and analog bus drivers to drive a multiplexed bus. Each frequency shifter shifts the frequency a different amount, so all the frequency-shifted signals can be added onto one bus. To prevent affecting the coils' field measurements, the bus contains no energy at the original coil signal frequencies. Alternatively, each coil may have its own integrated circuit.

Another suitable type of integrated circuit may employ a preamp, an analog-to-digital converter (ADC), and digital data encoding logic to drive a multiplexed data bus. Sine each coil has its own receiver electronics, this type of integrated circuit is characterized by fast measurement. With this implementation, the digital data bus is either on a radio-frequency carrier or an optical carrier. To avoid corrupting the field measurements, there is no energy in the data bus at the frequencies of the fields being measured.

If only one or a few receiver channel is/are used, the channel(s) are typically calibrated by applying known signals. If a channel-per-coil system is used, the channels are typically calibrated by putting a well-known field source in a well-defined location in the chamber.

Calibrating the chamber with well-known sources, also permits using wound coils (or other types of field sensors) instead of PCB coils. Typically, PCB coils do not themselves need to be calibrated because they are built precisely.

Methodological Implementation of Distortion-Compensation Using a Distortion-Mapping Chamber

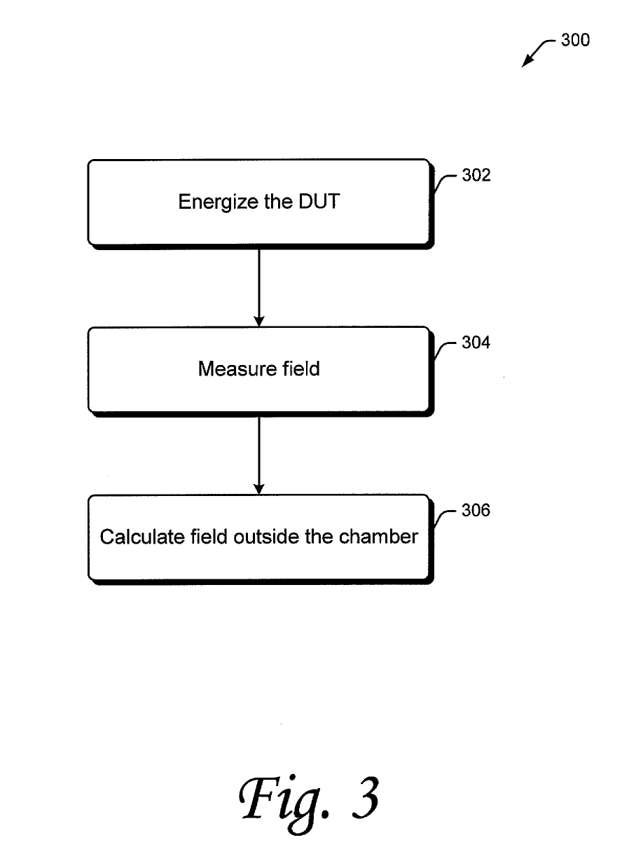

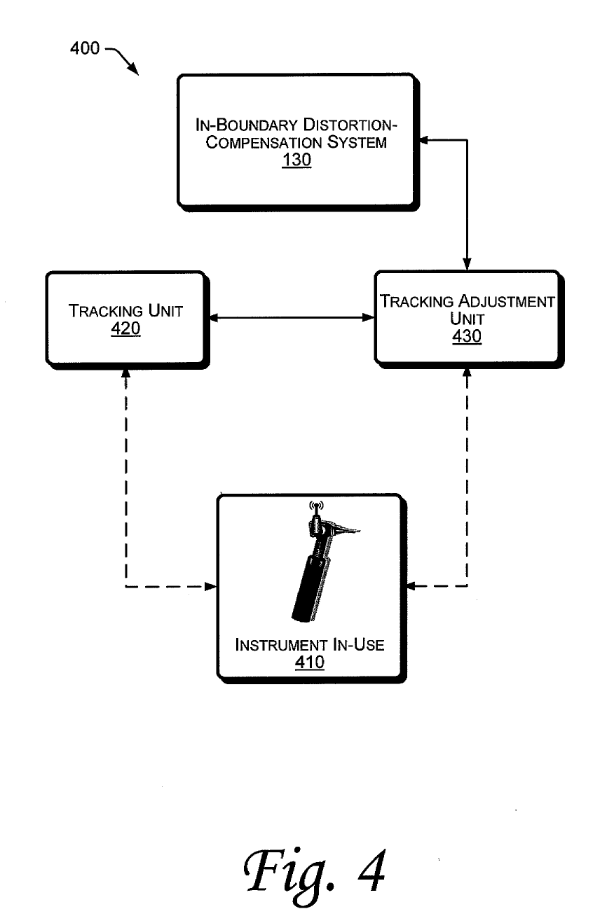

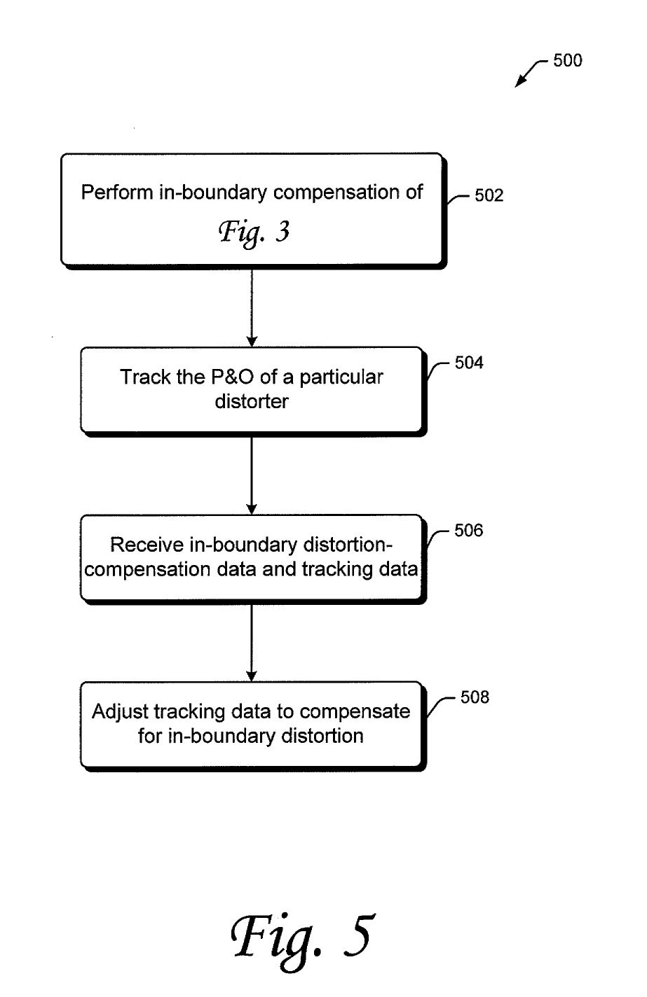

FIG. 3 shows method 300 for calibrating an in-boundary distortion-compensation system. This method 300 is performed by the one or more of the various components as depicted in FIGS. 1, 2A, and 2B. Furthermore, this method 300 may be performed in software, hardware, or a combination thereof. For ease of understanding, this method is delineated as separate steps represented as independent blocks in FIG. 3; however, these separately delineated steps should not be construed as necessarily order dependent in their performance. Additionally, for discussion purposes, the method 300 is described with reference to FIGS. 1, 2A, and/or 2B. Also for discussion purposes, particular components are indicated as performing particular functions; however, other components (or combinations of components) may perform the particular functions.

At 302 of FIG. 3, with the DUT 214 on the distorter 212 inside the distortion-mapping chamber (e.g., chambers 120, 210 and 220) and the chamber closed (such as it is at 220), the DUT is energized.

At 304, the field is measured by the coils on the walls of the distortion-mapping chamber (e.g., chambers 120, 210 and 220).

At 306, the distortion-calculation unit 110 receives these measurements and calculates the field outside the chamber accordingly. The calculations of the field outside the chamber (and thus outside the boundary surface) may be later employed to compensate for the in-boundary distortion caused by the distorter 212 on which the DUT 214 is attached when the distorter/DUT combination is later used in practice (such as in surgery and electromagnetic tracking of the DUT).

The field outside the chamber can then be calculated in various ways. Implementations described herein may employ the suitably adapted known field calculation approaches. For example, Green's functions may be used, but other known approaches may be used.

For example, an array-of-dipoles model or a current-element model may be employed. Since it is generally known approximately what the DUT 214 is, these models may be adjusted to fit the measured data. Such models are known to those of ordinary skill in the art. In the case of array-of-dipoles model, the strength, position, and orientation of each dipole are adjusted. In the case of the current-element model, the position, orientation, and length of each current element are adjusted, as the current is adjusted.

Furthermore, a mixture of both models may be used. This may be particularly appropriate for a DUT containing ferromagnetic materials such as ferrite.

If a current-element model is used, then one may display an image of the currents in the DUT. If a dipole-array model is used, then one may display an image of the dipoles in the DUT. These images would be of use in evaluating the contents of unknown DUTs.

### Exemplary Electromagnetic Tracking Using In-Boundary Distortion-Compensation

FIG. 4 shows, for the purpose of illustration, a simplified block-diagram of a electromagnetic tracking system 400 for use in improving position measurement accuracy in an electromagnetic (EM) tracker used in accordance with one or more implementations described herein. The electromagnetic tracking system 400 is an electromagnetic tracking system used with an image-guided surgery system.

As depicted, the electromagnetic tracking system 400 includes an instrument 410, a tracking unit 420, and a tracking adjustment unit 430. It also include the exemplary in-boundary distortion-compensation system 100 (which may be called a sub-system in this context) depicted in FIG. 1 and described above. The tracking unit 420 observes the tracking behavior of the instrument 410—which, for example, may be inside a patient during surgery. The tracking adjustment unit 430 attempts to improve or compensate for the tracking behavior of the instrument 410 to compensate for the in-boundary distortion caused by the instrument 410.

In an embodiment, the EM tracker includes a transmitter for transmitting a signal, a receiver for receiving the signal from the transmitter, and tracker electronics for analyzing the signal received by the receiver. The tracker electronics may be configured by software. The tracker electronics determines a position and/or an orientation of the instrument 410 in a tracking coordinate system based on information from a receiver and/or a transmitter. In an embodiment, the receiver is placed on the instrument 410 to determine the position and/or orientation of the instrument 410 in relation to the transmitter. In an alternative embodiment, the transmitter may be placed on the instrument 410 to determine the position and/or orientation of the instrument 410 in relation to the receiver. The EM navigation devices used in the EM tracker may be wired and/or wireless devices, for example (such as a wireless transmitter). A configuration of the EM tracker may be adjusted using information from the distortion-compensation system 100 to compensate for in-boundary distortion effects from instruments and operating environment.

In operation, the instrument 410 is tracked using the EM tracker or other EM navigation devices. The instrument 410 may be tracked in a physical EM tracker. The tracking unit 420 measures parameters, such as field, position, and orientation data, relating to the tracking of the instrument 410. The in-boundary distortion-compensation system 100 generates a distortion model and/or field map for the instrument 410. Then, the tracking adjustment unit 430 uses the model and/or map and any additional data from the tracking unit 420 to minimize the in-boundary distortion effects from the instrument 410.

The tracking unit 420 and the tracking adjustment unit 430 may be implemented in hardware and/or in software as separate units or a single unit. The tracking unit 420 and/or the tracking adjustment unit 430 may be integrated with the EM tracker or may be a separate system.

Furthermore, the tracking unit 420 and/or the tracking adjustment unit 430 may include one or more general-purpose or special-purpose computing system environments or configurations. Examples of well known computing systems, environments, and/or configurations that may be suitable for use include, but are not limited to, personal computers (PCs), server computers, hand-held or laptop devices, thin clients, thick clients, multiprocessor systems, microprocessor-based systems, programmable consumer electronics, wireless phones and equipment, general and special-purpose appliances, application-specific integrated circuits (ASICs), set top boxes, personal digital assistants (PDA), appliances, programmable consumer electronics, network PCs, minicomputers, mainframe computers, distributed computing environments that include any of the above systems or devices, and the like. The tracking unit 420 and/or the tracking adjustment unit 430 may one or more memories, which may be any available processor-readable media that is accessible by the units. The memory may be either volatile or non-volatile media. In addition, it may be either removable or non-removable media.

The instrument 410 may be, for example, any medical instrument with use in a medical activity, such as an orthopedic tool (an electric or pneumatic drill, for example), a catheter, scalpel, scope, debrider, stent, or other tool. The instrument 410 may generate or affect a magnetic field that causes distortion in readings of the EM tracker. Thus, it may be a distorter. An EM navigation device, such as a receiver or transmitter, is attached to the instrument 410. The distortion caused by the instrument may impact distortion and/or an effect of the distortion on tracking.

The tracking unit 420 tracks the instrument 410. More particularly, it tracks an EM tracker attached to the instrument 410. A receiver positioned on the instrument 410, a transmitter, and/or other sensors may be used to gather information about the instrument 410. The tracking unit 420 obtains magnetic field data for the instrument 410. The tracking unit 420 generates position and orientation data for the instrument 410 in a tracking coordinate system. However, the tracked position is likely to be distorted by the instrument 410 itself, which acts as an in-boundary distorter.

The tracking adjustment unit 430 adjusts or compensates for tracking behavior of the instrument 410. The tracking adjustment unit 430 uses the map, model, and/or other data from distortion-compensation system 100 to minimize distortion effects from the instrument 410. Using the calibration information from the in-boundary distortion-compensation system 100, the tracking adjustment unit 430 may modify, recalibrate, or reprogram the EM tracker to offset distortion effects from the instrument 410.

More particularly, the tracking behavior of the instrument 410 is adjusted or compensated in based upon measurements and calculations of the distortion-compensation system 100 and the distortion-compensation method 300 discussed above. These measurements and calculations are based upon a calibration performed on a particular distorter/DUT combination, which is illustrated in FIG. 2A as the distorter 212 and the DUT 214. For the calibration to apply, that particular distorter/DUT combination which was the subject of the measurements and calculations of the distortion-compensation system 100 and method 300 is the same as (or nearly identical to) the instrument in-use 410 (with its EM tracker attached thereto).

With this calibration, the tracking adjustment unit 430 may calibrate the EM tracker and/or the tracking adjustment unit 430 may adjust the position of a transmitter or receiver on the instrument 410 to compensate for or reduce in-boundary distortion in tracking.

Methodological Implementation of Electromagnetic Tracking Using In-Boundary Distortion-Compensation

FIG. 5 shows method 500 for distortion-handling EM tracking method. This method 500 is performed by the one or more of the various components as depicted in FIGS. 1, 2A, 2B, and/or 4. Furthermore, this method 500 may be performed in software, hardware, or a combination thereof. For ease of understanding, this method is delineated as separate steps represented as independent blocks in FIG. 5; however, these separately delineated steps should not be construed as necessarily order dependent in their performance. Additionally, for discussion purposes, the method 500 is described with reference to FIGS. 1, 2A, 2B, and/or 4. Also for discussion purposes, particular components are indicated as performing particular functions; however, other components (or combinations of components) may perform the particular functions.

At 502 of FIG. 5, the in-boundary distortion-compensation method 300 of FIG. 3 is performed and the in-boundary distortion caused by a particular distorter (e.g., instrument 410 with its EM tracker attached) is calculated.

At 504, the tracking unit 420 tracks the position and orientation (P&O) of the particular distorter (e.g., instrument 410 with its EM tracker attached)—which, for example, may be inside a patient during surgery.

At 506, the tracking adjustment unit 430 receives in-boundary distortion-compensation data from the distortion-compensation system 100 and tracking data from the tracking unit 420.

At 508, the tracking adjustment unit 430 adjusts the tracking data to compensate for the in-boundary distortion caused by the instrument 410. To make the adjustment, the tracking adjustment unit 430 uses the map, model, and/or other data from distortion-compensation system 100.

Other Applications, Implementations, and Details

The discussion herein focuses on the specifies of a medical tracking or navigational system, especially on used to track medical instruments in a patient's anatomy. However, the details of these described specifics are merely exemplary.

The functionality of the described implementations may and can be employed in variety of applications where it is desirable to accurately track the position of items other than medical instruments in a variety of applications. That is, a tracking system may be used in other settings where the position of an instrument in an object or an environment is difficult to accurately determine by visual inspection.

For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. The response signal is detected by a receiver. The signal emitted by the transponder may then be used to monitor the position of a person or object in a simulated environment.

Recall that, by reciprocity, the mutual inductance of two coils is the same, whichever coil is the transmitter and which is the receiver. Therefore, unless the context indicates otherwise, the reader should understand that when transmitters and receivers are discussed herein, the relative positioning and functionality of the receivers and transmitters may be swapped. Because of mutual inductance the functionality of the implementation with swapped receivers and transmitters remains the same as an implementation where there is no swapping of the receivers and transmitters.

Furthermore, the techniques, described herein, may be implemented in many ways, including (but not limited to) medical devices, medical systems, program modules, general- and special-purpose computing systems, network servers and equipment, dedicated electronics and hardware, and as part of one or more computer networks.

Although the one or more above-described implementations have been described in language specific to structural features and/or methodological steps, it is to be understood that other implementations may be practiced without the specific features or steps described. Rather, the specific features and steps are disclosed as preferred forms of one or more implementations.

1. An in-boundary distortion-compensation system comprising:
a distortion-mapping chamber configured to receive a device-under-test (DUT) with one or more associated distorters;
an in-boundary distortion-calculation unit configured to calculate the magnetic field outside of the distortion-mapping chamber to facilitate compensation for in-boundary distortion caused by the one or more associated distorters.

2. A system as recited in claim 1, wherein the distortion-mapping chamber forms a closed and finite boundary surface for a volume defined by the chamber itself.

3. A system as recited in claim 1, wherein the distortion-mapping chamber is a rectangular box having at least one closable opening into the volume inside the chamber.

4. A system as recited in claim 1, wherein the in-boundary distortion-calculation unit is further configured to employ Green's function for its calculation of the magnetic field outside of the distortion-mapping chamber.

5. A system as recited in claim 1, wherein the distortion-mapping chamber has one or more sides with a printed-circuit board having many non-overlapping or overlapping coils.

6. A system as recited in claim 1, wherein the distortion-mapping chamber has one or more sides with a printed-circuit board having many coils, the coils being associated with one or more switches, integrated circuits, preamps, frequency-shifting analog circuitry, analog bus drivers, analog-to-digital converters, and digital data encoding logic, or a combination thereof.

7. A system as recited in claim 1, wherein the distortion-mapping chamber forms a closed and finite boundary surface for a volume defined by the chamber itself and the in-boundary distortion-calculation unit is further configured to receive measurements of the magnetic field on the boundary surface.

8. An electromagnetic tracking system comprising:
An electromagnetic tracking unit configured to electromagnetically track a tracker attached to a distorter, the tracker being an object tracked by the electromagnetic tracking unit;
a tracking adjustment unit configured to adjust for in-boundary distortion caused by the distorter.

9. A system as recited in claim 8, wherein the tracker comprises one or more transmitters or receivers.

10. A system as recited in claim 8 further comprising an in-boundary distortion-compensation sub-system configured to analyze the in-boundary distortion of the distorter.

11. A system as recited in claim 8 further comprising an in-boundary distortion-compensation sub-system configured to analyze the in-boundary distortion of the distorter, wherein the tracking adjustment unit is further configured to obtain in-boundary distortion information from the in-boundary distortion-compensation sub-system and further configured to adjust for in-boundary distortion based upon the obtained information.

12. A system as recited in claim 8, wherein the distorter is a medical instrument.

13. A method comprising the acts of:
determining in-boundary distortion caused by a distorter on which a tracker is attached, the tracker is an object tracked by an electromagnetic tracking unit;
calculating in-boundary distortion-compensation data which compensates for the determined in-boundary distortion caused by the distorter;
tracking the tracker while the distorter is in-use and producing tracking data;
obtaining in-boundary distortion-compensation data and the tracking data;
adjusting the tracking data to for the determined in-boundary distortion caused by the distorter.

14. A method as recited in claim 13 further comprising measuring the magnetic field of a closed-and-finite boundary surface for a volume defined by a distortion-mapping chamber with the distorter on which the tracker is attached being located inside of the distortion-mapping chamber.

15. A method as recited in claim 13, wherein the calculating act employs Green's function.

16. A method as recited in claim 13, wherein the tracker comprises one or more transmitters or receivers.

17. A method as recited in claim 13, wherein the distorter is a medical instrument.
